(19) United States
(12) Patent Application Publication
(10) Pub. No.: US 2022/0370779 A1
(43) Pub. Date: Nov. 24, 2022

(54) NEEDLE IMPROVEMENTS FOR DRUG DELIVERY USING TATTOO MACHINES

(71) Applicants: Samia TRIGO ARBACHE, São José dos Campos (BR); Isabela TRIGO ARBACHE, São José dos Campos (BR)

(72) Inventors: Samia TRIGO ARBACHE, São José dos Campos (BR); Isabela TRIGO ARBACHE, São José dos Campos (BR)

(21) Appl. No.: 17/326,082

(22) Filed: May 20, 2021

(51) Int. Cl. A61M 37/00; A61F 9/00

(52) U.S. Cl. CPC A61M 37/0084; A61F 9/0017

(57) ABSTRACT

This invention privilege patent belongs to the field of medicine and surgical instruments, and is intended to provide a new route of drug administration through needles plated with specific metals, aiming at better drug distribution and absorption upon administration, more precisely in the eyeball and/or on the patient's skin; to which the product has been originally improved in order to increase its efficacy in relation to other means of drug delivery known in the medical field, especially in skin, mucous and ocular diseases; it is an improvement that allows drug delivery to the skin and/or subcutaneous tissue, by means of a duly modified tattoo machine, which instead of ink, uses drugs, as a product to be inserted in the skin; in addition, this invention can be used to administer drugs directly to a person's eyeball, offering great practicality and agility to surgical procedures.

### TECHNICAL FIELD OF INVENTION

This invention privilege patent belongs to the field of medicine and surgical instruments, and is intended to provide a new route of drug administration through needles plated with specific metals, aiming at better drug distribution and absorption upon administration, more precisely in the eyeball and/or on the patient's skin.

To which the product has been originally improved in order to increase its efficacy in relation to other means of drug delivery known in the medical field, especially in skin, mucous and ocular diseases.

It is an improvement that allows drug delivery to the skin and/or subcutaneous tissue, by means of a duly modified tattoo machine, which instead of ink, uses drugs, as a product to be inserted into the skin. In addition, this invention can be used to administer drugs directly to a person's eyeball, offering great practicality and agility to surgical procedures.

Also, the purpose of this application is to present improvements made to needles for drug delivery using tattoo machines, with low industrial manufacturing costs, combined with requirements of effectiveness, efficiency and safety, providing an additional treatment option which, unlike the usual methods, offers numerous possibilities and benefits to its users, making it a product of great acceptance in the medical sector.

### STATE OF THE TECHNIQUE

The oldest invasive route to administer active ingredients to the body is probably the technique used to tattoo the skin. There is great archaeological evidence that tattoos were already done in Egypt, between 4,000 and 2,000 BC, and also by Polynesian, Philippine, Indonesian and New Zealand natives.

In 1991, Otzi mummy was found in the Alps. It was a well-preserved male corpse, about 5,300 years old.

Otzi had 61 (sixty-one) tattoos coincidentally located on acupuncture points. Anthropologists believe that they probably had some therapeutic purpose.

Tattoos can be performed by various instruments. From iatrogenic tattoos (secondary to skin injection, mainly iron-based drugs), accidental tattoos (secondary to skin abrasion by asphalt), tattoos performed by solid needles or pointed instruments (known as amateur tattoos) and tattoos performed by machines (known as professional tattoos).

Tattoo machines are highly effective in injecting ink to the skin. This 1,000-year old technique gives machine operators an interesting route for administering ink to the skin.

The eye is a highly sophisticated and delicate structure. Simply stated, it is a device that resembles a camera, capturing static or moving images of the surroundings and transmitting them through the optic nerve to the central nervous system to be identified and interpreted. However, its sophistication contrasts with the inability of physicians to permeate its structures with drugs, fundamental to treat eye diseases. Drug bioavailability by transcorneal (through the cornea) and transconjunctival absorption is very low (e.g. eye drops), and the necessary therapeutic levels for eye disease treatment are not achieved.

Treatment of inflammatory, infectious and degenerative eye diseases are still a therapeutic challenge today. Oral, systemic, or topical drugs (eye drops) are unable to reach therapeutic doses at sites where there is infection or active inflammation, which makes current ophthalmological treatments ineffective in controlling infectious, inflammatory, and degenerative keratitis (inflammation of the cornea). Hence, the morbidity (number for patients affected by a particular disease) of ophthalmological diseases is high, forcing ophthalmologists to perform surgical interventions with high costs for the healthcare system (e.g. penetrating corneal transplants). The social and economic impact of “incurable” eye diseases is significant, occasionally leading to tragic outcomes such as blindness, in some cases evolving to evisceration (surgical removal of the eyeball) by purulent endophthalmitis (eye infection) secondary to keratitis (inflammation of the cornea). Conjunctival and scleral disorders are another therapeutic challenge, since both are highly immunoreactive means, sites of hypersensitivity reactions difficult to be clinically controlled, and autoimmune diseases affecting the vascular system with high morbidity levels.

Therefore, in this scenario, designing techniques to easily inject drugs to the eyeball is fundamental.

Drug injections to the eye using a syringe is a common method employed by ophthalmologists, the most commonly performed routes being intracorneal (corneal injections) or intraocular (injections into the eye).

Intracorneal and intraocular injections with syringes are valid procedures accepted by the global scientific community because they effectively inject drugs into the eyeball. However, these procedures are technically dependent because they are subject to physician's finger pressure variability when pressing the syringe plunger, with consequent irregular distribution of the drug in the area to be treated, with the possibility of sequelae. If the pressure exerted on the syringe is exaggerated, it may cause irregular distribution of the drug and adverse effects by a compressive effect.

In summary, ophthalmologists have technical limitations to treat the eyeball with drugs.

By retrospectively reviewing the topic, we have identified in the literature references to ocular interventions performed by Galen (150 AD) and Aetius (450 AD) intended to pigment-cover corneas with leukoma (white opacity of the cornea that resembles scars and blindness, usually secondary to ulcers or corneal trauma) using inks similar to the ones used for tattooing.

The corneal pigmentation technique was again performed in 1870 by the oculoplastic surgeon Louis Von Wecker, laying the foundations of the modern micro pigmentation technique for corneal scars in eyes without sight, widely known in current medicine, being improved for the use of multiple needles and different pigments, as in the series of publications by Archer et al (Utrecht, 1874).

Until recently, corneal dermopigmentation was indicated for corneal leukomas only in eyes whose visual prognosis was extremely reserved, especially in patients who did not adapt to the use of cosmetic contact lens. In these cases, inserting pigments through needles to cover disfiguring leukomas was quite successful.

Recently, an equipment has been described that impact-inserts a tiny biodegradable tip into the cornea that gradually releases drug. The tip of this device is pre-formed with the drug and inserted under pressure at the base of the needle.

Another technique includes introducing drug-coated needles, always with the same purpose, to deliver drugs to the cornea.

Aiming at introducing a new product to the market, a state of the art search was carried out in a specialized database and the following processes were found:

PI 9900009-1—“TRANSFER DEVICE OF AT LEAST ONE ACTIVE PRODUCT INSIDE THE HUMAN EYEBALL BY IONTOPHORESIS”, this invention relates to a transfer device of at least one active product, specifically a drug, to the eyeball by means of iontophoresis, comprising an active product reservoir (15), and capable of being applied over the eye of a patient. The reservoir (15) has at least one surface active electrode (11) placed across an eye tissue located at the periphery of the cornea (C), a return electrode (12) and a current generator. The return electrode (12) is preferably in contact with the patient's partially closed eyelids (22, 24).

BR 11 2015 025358-0—“EYE DROPPER AND METHOD FOR ADMINISTERING LIQUID DROPS TO EYE SURFACE”, an eye dropper (2, 102) with improved stability is provided for delivering a volume of liquid on the surface of the eye (57). The eye dropper (2, 102) includes a body (4, 104) having a reservoir body (18, 118), a first rod (14, 114), a second rod (16, 116), and a mouthpiece (26, 126). The mouthpiece (26, 126) is in fluid communication with the inner cavity (24, 124) of the reservoir body (18, 118) and is projected from the outer surface (20, 120) of the reservoir body (18, 118) between the first and second rods (14, 16, 114, 116). The first and second rods (14, 16, 114, 116) are separated by a distance which is at least equal to the height of the eye dropper (2, 102). In preferred embodiments, the distance between the first and second rod (14, 16, 114, 116) is greater than the height of the reservoir body (18, 118).

BR 11 2017 008658-1—“SYRINGE AND SYRINGE PREPARATION METHOD”, the present invention relates to a syringe (18) having a longitudinal body (28) with an interior (228) containing a pharmaceutical substance, a needle connected to a longitudinal end of the body (228) and a rigid needle guard (38) surrounding the needle. The rigid needle guard (38) is essentially impermeable to water vapor. The syringe (18), according to the invention, prevents needle clogging and, thus, allows the adequate delivery of pharmaceuticals, particularly by subcutaneous, intramuscular or ocular injection.

BR 11 2015 027762-4—“OCULAR INJECTION APPARATUS AND METHODS”, it is an apparatus including a housing coupled to a drug container which is coupled to a needle. An injection assembly is located within the housing and includes a power storage member and actuating rod. A distal end portion of the actuating rod is located inside the drug container. The energy storage member may produce a force on a proximal end portion of the actuating rod sufficient to move the distal end portion of the actuating rod inside the drug container. This may carry at least a portion of a substance from the drug container through the needle when a distal tip of the needle is located within a first region of a target location. The force is insufficient to move the distal end portion of the actuating rod inside the drug container when the distal tip of the needle is located within a second region of the target location.

In addition, various previous state of the technique are known which are related to ink infusion on the skin, nothing specific for drug infusion, the identifications and summaries of which are listed below.

U.S. Pat. No. 6,505,530 B2—“INK DELIVERY DEVICE FOR TATTOOING OR PERMANENT MAKEUP”, an ink application device for tattooing or permanent makeup that includes a handle, a needle unit, a needle, a needle tip and a protective case. The device further comprises at least two modules connected to one another. One of the modules is formed as a reusable base module with an integrated needle unit, and the other module is a sterile disposable module in which all components of the handheld device can be infected by a patient's body fluids. The device can have its construct divided in two or three distinct modules: a basic module, a disposable or hygienic module, and an optional ink module. The base module corresponds to the handle which includes the needle unit and is designed so that the hygiene module can be linked and connected securely to the integrated needle unit.

US 20050010236 A1—“INK DEVICE FOR TATTOOING OR PERMANENT MAKEUP”, the invention relates to an apparatus used for permanent tattooing or a composition, comprising a disposable detachable module arranged in a handpiece, a needle held in a end member on a needle shaft and supported for movement of a needle guide formed in the disposable module and the other end extending through an opening of the discharging mouthpiece to the color needle, and a drive mechanism comprised of several structural elements and coupled to the needle axis to effect needle back and forth movements, said plurality of structural elements of the drive mechanism of the unit and that comprises means for coupling the drive devices to the needle rod. At least a portion of the coupling means is located in a disposable and detachable module of the handpiece together with the disposable module.

US 20100036317 A—“PORTABLE DEVICE FOR HUMAN OR ANIMAL SKIN PUNCTURING SITE, DRIVE UNIT, NEEDLE UNIT AND A COUPLING METHOD”, the invention relates to a portable device for a human or an animal skin puncture site, in particular for the introduction of an active substance or for the application of a permanent tattoo or of a composition comprising a drive unit, set up to move the repetitive unit, and a needle unit, which comprises a needle device and is set up to be coupled with the drive unit so that movement of the repetitive unit for needle extension and retraction in the device can be coupled to the latter; said drive unit has an actuation member on it, which is functionally displaceable, coupled to a needle attachment device, and said needle unit having therein a functional member that is assigned to the drive member, said drive member and said functional member are set up to move the drive member from a first displacement position to a second displacement position by means of the functional attachment member designated as the needle unit for the drive unit, thus moving the needle attachment device to an engaging position in which the needle can be coupled to the needle attachment device for the purpose of operation. Further, the invention relates to a method for coupling a needle unit to a drive unit of a hand device, a drive unit for a hand device, as well as a needle unit for a portable device.

U.S. Pat. No. 7,340,980 B2—“TATTOO MACHINE”, a tattoo machine comprising at least one elongated hollow body, within which there is a cavity for containing a staining liquid connected to the outside by an orifice at one of the ends of the hollow body, a needle that can slide alternatively along the holes, and elements for the passage of the needle, between a first end position, where the needle projects at least partially from the hollow body, and a second end position, where the needle is fully accommodated within the hollow body, the elements of the movement comprising a motor which can be associated with the needle, with interposition of movement transmission elements supplied with a flexible cable, at a first end of which may be associated with the motor and a second end that may be associated with the needle.

US 20050277973 A—“ULTRASONIC TATTOOING EQUIPMENT”, an ultrasound device for applying skin pigmentation in which the device moves the needle to apply the pigment in an ultrasound band. The device may be self-contained, self-powered, or powered externally. The invention may also include replaceable needles, and/or a pigment reservoir. The device may also have course length adjustments to prevent stroke. The mechanism that moves the needle can be a piezo unit, a linear unit or a mechanism of solenoid type or an actuator unit.

US 20120221036 A—“TATTOOING DEVICE”, a method and apparatus for marking a treatment isocenter in a patient's body. One embodiment includes a base, including alignment marks, a marker disposed on the base and positioned relative to the isocenter marking reference on the patient's body and an actuator for triggering the marker and causing a mark indicating the isocenter in the patient's body. The actuator may include a key and a spring attached to the marker. Actuator compressing causes the marker to pass through an ink well before piercing the patient's skin.

The major problem of skin treatment methods using topical drugs, on the one hand, has to do with the body having difficulty absorbing topical drugs, since the skin, as a human organ, is a natural protection barrier that prevents the desired drug absorption.

On the other hand, injectable skin treatment methods deliver excessive, irregular and/or deficient amounts of drug at a single point, making absorption disproportionate as to the amount of drug to be absorbed by the total area of the skin to be treated.

A high-frequency fractionating device called “LEGATTO” has been designed, which proposes a solution similar to the method in the presentation “to perfuse” drugs on the skin, but it is a completely different technique in which radio-frequency equipment makes micro perforations on the skin, delivers the drug to the skin and subsequently applies an ultrasound handpiece to force absorption.

It is found that the above-mentioned documents and the methods described in the prior art, although belonging to the same field, do not have the same technical characteristics of the object hereby improved, thus ensuring it meets the necessary legal requirements for patentability.

### BRIEF DESCRIPTION OF THE INVENTION

In order to overcome the drawbacks and limitations present in the state of the art, the inventor has created and developed the present invention by devising needles for injecting drugs into the corneal stroma and/or into eye structures and into the dermis or subcutaneous tissue through tattoo machines, in which the needles make a back and forth movement.

Tattooing, that is, the insertion of chemicals into the skin by means of needles, is a 1,000-year old technique. Eye tattooing was described by Galen in 150 AD and forgotten by humankind. Ocular pigmentation was rediscovered by De Wecker, fourteen centuries later. Afterwards, several studies described drug delivery into the eyeball using needles impregnated with drugs.

Backed up by the history of tattoos documented thousands of years ago and by the scientific documentation researched, pigments and drugs were injected into the eyes of rabbits for testing purposes after ethical approval from an Animal Ethics Committee. Subsequently, after ink and drug delivery, anatomical and pathological examinations confirmed that the injection of chemicals into the eyeball is an effective procedure, without any adverse effect being detected.

The main advantage of injecting drugs into the eyeball using tattoo machines with specific needles is that it is possible to inject drugs in small quantities with homogeneity that cannot be achieved when the same procedure is performed by a physician using syringes and needles, since application is irregular and technically-dependent.

In addition, the present patent has also been developed on the basis of proven drug skin absorption research which has resulted in the adaptation of a tattoo machine, conveniently configured and arranged to allow drug delivery to be better distributed under the skin, with unparalleled effectiveness and versatility, without the drawbacks already mentioned.

In existing needle models already manufactured with the drug inserted into the needle, whether in its structure or by coating, physicians are not able to choose the drug and its strength.

In the technique described herein, physicians can choose specific drugs for the eye/skin disease and also decrease or increase the strength of the drug to be injected.

It is understood that the present invention developed for the application of the technique in question offers excellent results, thus incorporating an innovative method that makes it possible to minimize potential inconveniences.

### BRIEF DESCRIPTION OF THE FIGURES

To complement the present description in order to obtain a better understanding of the features of the present invention and in accordance with a preferred practical embodiment thereof, a set of figures, attached, accompany the description, where operation has been represented with examples that are not limited:

FIG. 1 shows the detail of needles with tips aligned with the curvature of the eye;

FIG. 2 shows the detail of needle tip oval arrangement;

FIG. 3 shows the detail of the needles seen from below;

FIG. 4 shows a perspective view of the needles;

FIG. 5 shows a perspective view of a set of tattoo needles used in the method for drug micro infusion on the skin through a tattoo machine.

FIG. 6 shows a perspective view of a tattoo tip used in drug micro infusion through a tattoo machine.

FIG. 7 shows a side view in transparency of the lower portion of a tip used in drug micro infusion through a tattoo machine.

FIG. 8 shows a detailed view of the application in drug micro infusion on the skin through a tattoo machine.

### DETAILED DESCRIPTION OF THE INVENTION

According to the above-mentioned drawings, the present invention relates to metal-plated needles (1) for injecting drugs into the eyeball (8) and/or the skin (9) by means of tattoo machines to make eye and/or skin disease treatment more effective, more efficient and safer.

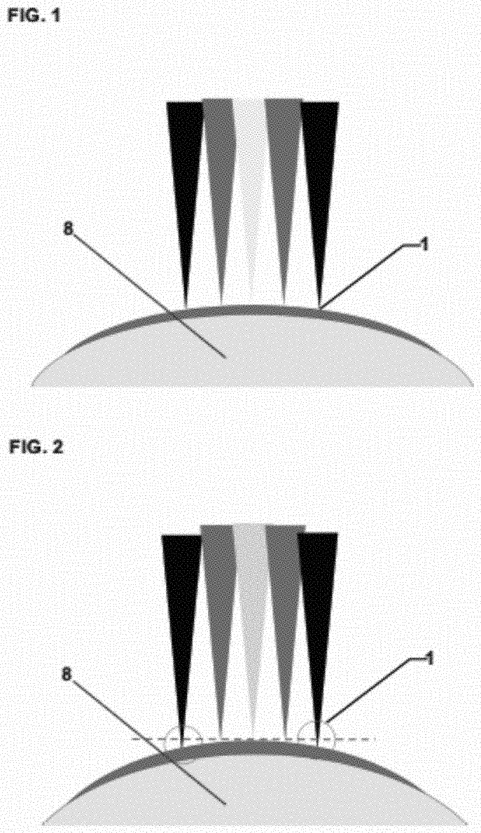

The principle of the invention is very similar to ancient tattoo techniques. In a drug-loaded cartridge (6), with the drug strength being chosen by a physician, the needles (1), in a back and forth movement, deliver the drug to the corneal stroma (8) and/or eye structures (8) and/or the skin (9). It should be pointed out that, because of the round anatomical structure, the needles (1), as a whole, should be arranged so that the tips (1) are aligned with the curvature of the eye (8) and/or the skin (9), as shown in FIGS. 1, 2 and 8.

Drug is delivered by means of an adapted tattoo machine which, instead of ink, uses specific drugs, the tattoo machine has a disposable single-piece tip (5) and a drug container (6) with a mouthpiece projected outwardly and deeply with variable dosing holes, positioned approximately mid-height of the tip (5) and which discharges the drug by shearing onto oscillating needle bundles (7).

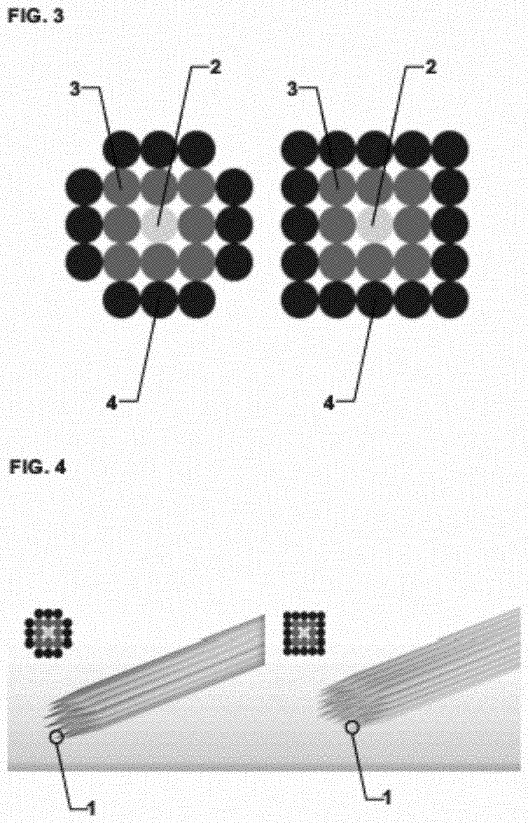

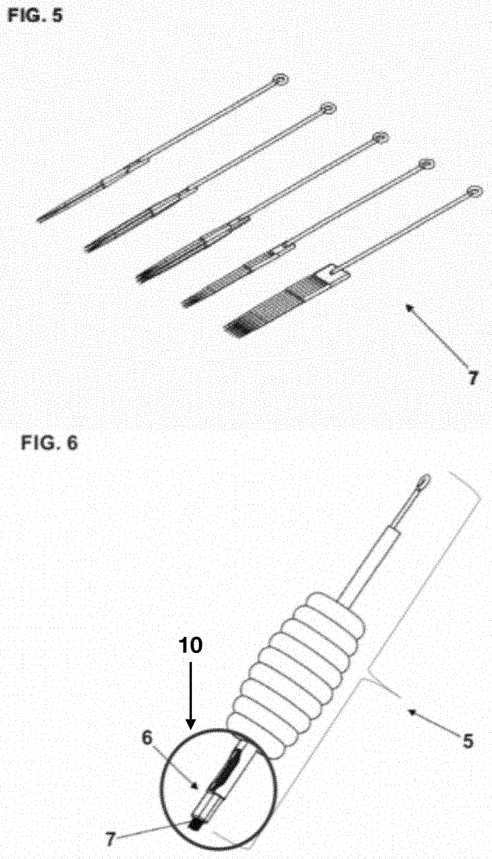

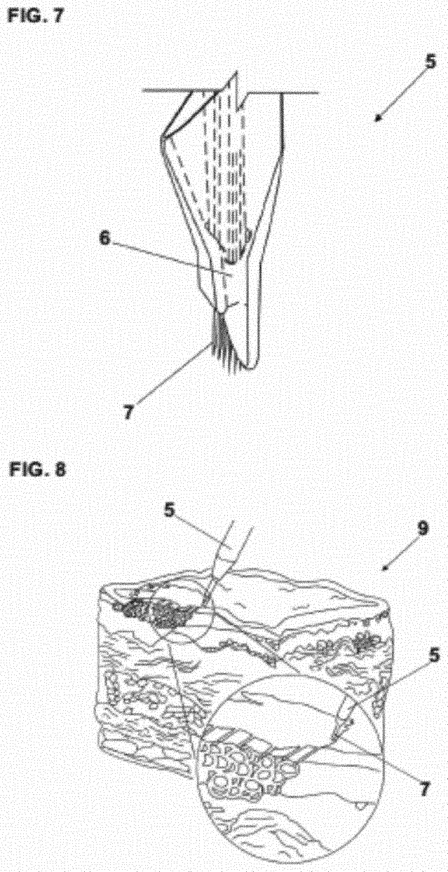

According to FIG. 3, the needles (1) are arranged in a group, the central needle (2) being more retracted, the adjacent needles (3) less retracted and the externally arranged needles (4) are more exposed, so that they align with the curvature of the eye (8).

The needles (1) should be plated with specific metals, for example, gold for the following reasons:
Gold is less likely to cause allergic reactions in the area to be treated;
Gold has bactericidal properties; and
Gold plating makes the surface of the needle smoother.

The invention is susceptible to variations that may occur in needle morphology (number of needles (1), diameter and adjustments in tip curvature) and metal coating.

Certainly, when the present invention is put into practice, modifications can be made with respect to certain details of construction and shape, without departing from the fundamental principles that are clearly substantiated in the claim, being understood that the terminology employed was not intended to be limiting.

This is a descriptive report of a completely new concept which, as it can be seen from the analyzes carried out, shows new technical and functional characteristics.

For the advantages being offered, and yet, because it has truly innovative features that meet all the requirements of novelty and originality in the genre, the present invention meets the conditions necessary to merit the patent of invention.

1. A drug delivery system using a tattoo machine comprising:
a removable cartridge including a needle bundle tip and a drug container having an opening configured to be loaded with a drug, said needle bundle tip comprises a plurality of tattoo needles arranged together so that the tips of said plurality of tattoo needles are aligned with either a curvature of an eye or a contour of the skin of a patient, wherein the drug is discharged by shearing onto oscillating said plurality of tattoo needles.

2. The drug delivery system of claim 1, wherein a strength of said drug is selected by a physician.

3. The drug delivery system of claim 1, wherein the drug is delivered to one of: a corneal stroma, an eye structure and a patient skin by a back and forth movement of said plurality of tattoo needles.

4. The drug delivery system of claim 1, wherein the opening of said drug container has with a mouthpiece projected outwardly with variable dosing holes positioned approximately midway of said cartridge.

5. The drug delivery system of claim 1, wherein the plurality of tattoo needles are arranged so that a central needle portion is retracted, an adjacent needle portion is less retracted than said central needle portion and an externally arranged needle portion is more exposed than said adjacent needle portion so that plurality of tattoo needles tips is aligned with the curvature of the eye.

6. The drug delivery system of claim 1, wherein said drug container is a removable, replaceable and disposable unit.

7. The drug delivery system of claim 1, wherein said plurality of tattoo needles is plated with a metal.

8. The drug delivery system of claim 1, wherein said plurality of tattoo needles is plated with a gold.

9. A Needle for injecting drugs into the eyeball and/or the skin, by means of tattoo machines, with a cartridge loaded with a specific drug at a strength chosen by the physician, characterized with the cartridge being loaded with a specific drug at a strength chosen by the physician, the needles in a back and forth movement, deliver the drug to the corneal stroma and/or eye structures and/or the skin; the needles, as a whole, should be arranged so that the tips are aligned with the curvature of the eye and/or the skin; the drug is delivered by means of an adapted tattoo machine having a disposable single-piece tip having a drug container, with a mouthpiece projected outwardly and deeply with variable dosing holes positioned approximately mid-height of the tip and which discharges the drug by shearing onto oscillating needle bundles; the needles are arranged in a group, the central needle being more retracted, the adjacent needles less retracted and the externally arranged needles are more exposed so that they align with the curvature of the eye.
